(19) United States
(12) Patent Application Publication
(10) Pub. No.: US 2005/0221321 A1
(43) Pub. Date: Oct. 6, 2005

(54) Method of detecting rheumatoid arthritis by detecting overexpression of wnt

(76) Inventor: Kazushi Imai, Shinjuku-ku (JP)

Correspondence Address: SHERMAN & SHALLOWAY, 415 NORTH ALFRED STREET, ALEXANDRIA, VA, 22314, US

(21) Appl. No.: 10/511,910

(22) PCT Filed: Apr. 25, 2003
(86) PCT No.: PCT/JP03/05358

§ 371 Date: Oct. 20, 2004

(30) Foreign Application Priority Data
May 2, 2002 (JP) 2002-130883

(51) Int. Cl. C12Q 1/68

(52) U.S. Cl. 435/6

(57) ABSTRACT

A convenient and highly accurate pre-examination of detecting rheumatoid arthritis wherein at least the upregulation of expression of WNT at least the upregulation of expression of WNT10B and at least the regulated expression of FRP1 in joint synovial liquid, joint synovial tissue or peripheral blood are detected in parallel.

### FIELD OF THE INVENTION

The present invention relates to the method for detection of rheumatoid arthritis by monitoring the upregulation of expression of WNT, especially WNT 10B, in joint synovial fluid or in peripheral blood by a reverse transcription (RT) PCR analysis. In addition, it includes the method for detection of secreted frizzled-related protein (FRP) in parallel with that of WNT.

### BACK GROUND OF THE ART

For the diagnosis of rheumatoid arthritis (RA), the criteria which was advocated by the American College of Rheumatology is adapted worldwide. The criteria consists of, 1) stiffness which continues more than one hour in the morning, 2) swelling in more than three joints, 3) swelling and deformity in joints of the hand, 4) symmetrical swelling of the joint, 5) aberration of the hand joints verified by radiography, 6) subcutaneous nodules, and 7) positive reaction of rheumatoid factor by blood examination. To diagnose patients as RA, it is required to ascertain more than 4 criteria, and the criteria 1)-4) should continue over 6 weeks. Since disease onset is insidious in most cases and the diagnosis criteria is largely depending on clinical findings, several months or years can elapse before a firm diagnosis can be ascertained. The delay of the diagnosis reduces the effectiveness of conservative therapies, and urge patients to burden with the mental, physical and economical demands.

As a biochemical examination, measuring the serum levels of rheumatoid factor are widely utilized to predict the onset of RA. However, it is a controversial issue that the rheumatoid factor can reflect the onset because of high frequency of false-positive and false-negative cases.

Therefore, there is an urgent need to develop a novel strategy that makes possible to diagnose patients suffered from RA in the early stage with high specificity (http://www.rheuma-net.or.jp/).

The WNT family was cloned as an oncogenic gene family and consists of 19 paralogues in the human genome. It stimulates an expression of c-Myc and Cyclin D1 and enhances proliferation of tumor cells and endothelial cells (Wright, M., Aikawa, M. Szto, W. and Papkoff, J. Identification of a WNT-responsive signal transduction pathway in primary endothelial cells. Biochem. Biophys. Res. Commun. 263:384-388, 1999). Recently, it is reported that RA synovium increase the expression of WNT1 and WNT5A, and the production of inflammatory cytokines including Interleukin 6, 8, and 15 (refer to Sen. M., Lauterbach, K., EI-Gabawy, H., Firestein, G. S., Corr, M. and Carson, D. A. Expression and function of wingless and frizzled homologs in rheumatoid arthritis. Proc. Natl. ACAD. Sci. USA. 97: 2791-2796, 2000). In contrast, the synovial tissue from osteoarthritic (OA) patients did not express WNT1 and WNT5A.

Further, in Japanese Patent Laid-Open Publication 11-113580, specifically, in lines 1-3 of the fourth column, the art regarding diagnostic assay for diseases relating WNT-11 activity or level is referred, and from [0033] to [0034] said diagnostic assay is explained. However, there is no refer explaining that the RA specific diagnosis is possible by detecting the RA specific expression of WNT, especially WNT10B. Further, the expression of WNT11 at RA joint synovium, and the diagnostic assay by WNT11 detection is not included in the RA specific diagnosis method of the present invention.

The subject of the present invention is the providing of RA specific diagnosis method which detects the upregulation of WNT expression in the joint synovial tissue and fluids and peripheral blood, and enable us to diagnose RA in the early stage and to start the preventive therapeutics.

WNT is expressed in various types of developing organs and plays a pivotal role in the organogenesis and morphogenesis of embryonic stage (Moon, R. A. Brown, J. D. and Torres, M. WNTs modulate cell fate and behavior during vertebrate development. Trends Genet. 13: 157-162, 1997.). However, since the expression ceases after the completion of organogenesis, it is predicted that WNT does not have a predominant role in maintaining the integrity of adult organs. Therefore, aberrant activation of the WNT expression may contribute to cause or stimulate the pathological changes observed in RA synovium.

The inventor of the present invention, has clarified and compared the profiling of expression pattern of WNTs and FRPs, specific inhibitors of the WNT family, between patients with RA in which the pathological changes of synovium is associated and with OA which is not related to the synovial abnormalities. WNTIOB was specifically expressed in RA synovium but other WNT members were less frequently or negligibly expressed in RA and OA synovium. In addition, WNT10B specifically localized to synovial surface cells and endothelial cells in RA tissues.

On the contrary, FRPs were specifically expressed OA synovium, especially, FRP1 expression was identified in all of the OA tissues, and localized in synovial surface cells and endothelial cells, which were identical to that of RA tissues. Above data predicts that the expression of FRPs may prevent the action of WNT and the subsequent pathological activation.

Since the expression of WNT10B and FRP1 discriminates the nature of RA and OA synovium, a novel RA-specific diagnosis method could be established by detecting the presence of WNT and FRP, in particular WNT10B and FRP1, in joint synovial fluid or peripheral blood, and the subject of the present invention can be dissolved.

### DISCLOSURE OF THE INVENTION

The present invention is (1) a method to detect rheumatoid arthritis by detecting at least the upregulation of expression of WNT10B in joint synovial fluid, in joint synovial tissue or in peripheral blood. Desirably, the present invention is (2) the method to detect rheumatoid arthritis of (1), wherein at least the upregulation of expression of WNT10B is detected by RT-PCR analysis. More desirably, the present invention is (3) the method to detect rheumatoid arthritis of (1) or (2), wherein at least inhabitation of expression of FRP is detected in parallel. Furthermore desirably, the present invention is (4) the method to detect rheumatoid arthritis of (3), wherein at least an inhabitation of expression of FRP is detected in parallel.

### BRIEF ILLUSTRATION OF THE DRAWING

FIG. 1 shows the expression of WNT gene by RT-PCR analysis. Lanes 1-5 indicate the expression of WNT3, WNT5A, WNT10B and WNT14 in RA synovial tissue, and lanes 6-9 indicate expression of WNT3, WNT5A, WNT10B and WNT14 in OA synovial tissue.

FIG. 2 shows the expression of FRP gene by RT-PCR analysis. Lanes 1-5 indicate the expression of FRP1, FRP2, FRP3 and FRP4 and FRP5 in RA synovial tissue, and lanes 6-9 indicate expression of FRP1, FRP2, FRP3, FRP4 and FRP5 in OA synovial tissue.

### DESCRIPTION OF THE PREFERRED EMBODYMENT

The present invention will be illustrated in more in detail.

1. Profiling the expression pattern of WNTs and their specific inhibitors, FRPs, which were specifically detected in RA and OA synovium, respectively.
(1) Total RNA was isolated from patients suffered from RA (five cases) or OA (four cases) undergoing total joint replacement surgery according to the Chomczynski and Sacchi method. To prevent contamination of genomic DNA, RNA samples were treated by RNase-free DNaseI at 37° C. for 30 minutes.

(2) 1^{st} strand cDNA (50 μl) was synthesized from 5 μg of total RNA using oligo d(T) primer (Gibco BRL, Giathusberg, Germany) and reverse transcriptase (SuperScriptII, Gibco BRL). After incubation of 1 μl of cDNA at 94° C. for 5 minutes, PCR reaction was carried out using gene-specific primer sets, novel base series indicated in Table 1, and Taq DNA polymerase (Gibco BRL). Denaturing was carried out on 50 μl of each reaction mixtures at 94° C. for 30 seconds, at proper annealing temperature indicated in Table 1 for 30 seconds, and extension at 72° C. for one minute. These procedures were set as the one cycle, and additional 29 cycles were repeated. The reaction mixture was analyzed by gel electrophoresis on 2% agarose gels and the presence of gene-specific amplification was confirmed.

TABLE 1

| | | annealing |
|---|---|---|
| | | temper- |
| Primer | series | ature |
| WNT1 | (forward) 5′-TCCTGCTCAGAAGGTTCCAT | 54 |
| | (reverse) 5′-GCTGTACGTGCAGAAGTTGG | |
| WNT2 | (forward) 5′-CTGTATCAGGGACCGAGAGG | 51 |
| | (reverse) 5′-CTGTATCAGGGACCGAGAGG | |
| WNT2B | (forward) 5′-ACTGAGTGTGTGCAGCTGTG | 54 |
| | (reverse) 5′-TGATGTCTTGCTGCAGACAC | |
| WNT3 | (forward) 5′-ACTTCGGCGTGTTAGTCTCC | 54 |
| | (reverse) 5′-ATTTTTCCTTCCGCTTCTCC | |
| WNT4 | (forward) 5′-TTGAGGAGTGCCACTACCAG | 54 |
| | (reverse) 5′-TTGAACTGTGCGTTGCGTGG | |
| WNT5A | (forward) 5′-CAGTTCAAGACCGTGCAGAC | 58 |
| | (reverse) 5′-TGGAACCTACCCATCCCATA | |
| WNT5B | (forward) 5′-GTGCTGCTTCGTCAGGTGTA | 54 |
| | (reverse) 5′-CGAGGTTGAAGCTGAGTTCC | |
| WNT6 | (forward) 5′-CAACTGCACAACAACGAGGC | 54 |
| | (reverse) 5′-GTACTACGCAGCACCAGTGG | |
| WNT7A | (forward) 5′-GAGAAGCAAGGCCAGTACCA | 54 |
| | (reverse) 5′-ACAGCACATGAGGTCACAGC | |
| WNT8A | (forward) 5′-ACATGCTATCAGCTCTGCTG | 54 |
| | (reverse) 5′-AAAGATCAGTTCCGCCTCTG | |
| WNT8B | (forward) 5′-GAAAGTGGCAAGCTTTGGAG | 54 |
| | (reverse) 5′-GAAAGTGGCAAGCTTTGGAG | |
| WNT10A | (forward) 5′-AATGAGGCTTCACAACAACC | 54 |
| | (reverse) 5′-TCATGTGGTCCAATCTCCTC | |
| WNT10B | (forward) 5′-CTTCATTGATACCCACAACC | 58 |
| | (reverse) 5′-ATTGTTGGGGAGAAGGCTAC | |
| WNT11 | (forward) 5′-TGACCTCAAGACCCGATACC | 54 |
| | (reverse) 5′-CAAGTGAAGGCAAAGCACAA | |
| WNT14 | (forward) 5′-AAGATGGTGCCAACTTCACC | 58 |
| | (reverse) 5′-TAAGGAACCAGCCAGGACAC | |
| WNT16 | (forward) 5′-GTGACACCACCTTGCAGAAC | 54 |
| | (reverse) 5′-ACCCTCTGATGTACGGTTGC | |
| FRP1 | (forward) 5′-CTTGTTCTTGCAGCATTCCC | 54 |
| | (reverse) 5′-AGAGAAGGCAATGCCTCTCC | |
| FRP2 | (forward) 5′-AAAGACAGCTTGCAGTGCAC | 54 |
| | (reverse) 5′-TGTTATGACAACCTCAGTGG | |
| FRP3 | (forward) 5′-CATTGACTTCCAGCACGAGC | 56 |
| | (reverse) 5′-ACGAAGCTTCATATCCCAGC | |
| FRP4 | (forward) 5′-AGAGGAGTGGCTGCAATGAG | 58 |
| | (reverse) 5′-TGGCCTTACATAGGCTGTCC | |
| FRP5 | (forward) 5′-AAGTGGATGGACAGCTGCTG | 54 |
| | (reverse) 5′-TACTTTCTGAGACCCTGAGG | |
| GAPDH | (forward) 5′-GTCAGTGGTGGACCTGACCT | 52 |
| | (reverse) 5′-AGGGGAGCTTCAGTGTGGTG | |

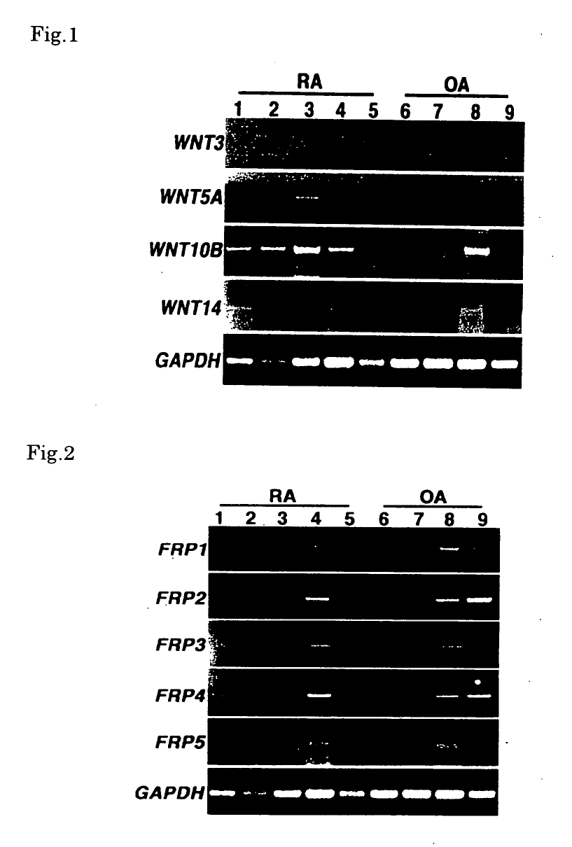

WNT10B was detected in four of five RA synovium but limited to one of four OA cases by RT-PCR. Little or no expression of other WNT members was detected. Representative results by RT-PCR were shown in FIG. 1. FRP1 was detected in all of four OA cases analyzed, FRP2 and FRP4 in 2/4 cases, FRP3 and FRF5 were observed in 1/4 case. In RA samples, each FRP gene was expressed in 1/5 case or not expressed. Expression of FRPs was represented in FIG. 2. Table 2 summarized the results of RT-PCR: +; presence of expression, −; absence of expression, ND; not determined.

TABLE 2

| | RA | | | | | OA | | | |
|---|---|---|---|---|---|---|---|---|---|
| | 1 | 2 | 3 | 4 | 5 | 6 | 7 | 8 | 9 |
| WNT1 | − | − | − | − | − | − | ND | − | − |
| WNT2 | − | − | − | − | − | − | ND | − | − |
| WNT2B | − | − | − | − | − | − | ND | − | − |
| WNT3 | − | − | − | − | − | − | − | − | − |
| WNT4 | − | − | − | − | − | − | ND | − | − |
| WNT5A | − | − | + | − | − | − | − | − | − |
| WNT5B | − | − | − | + | − | − | ND | + | − |
| WNT6 | − | − | − | − | − | − | ND | − | − |
| WNT7A | − | − | − | − | − | − | ND | − | − |
| WNT8A | − | − | − | − | − | − | ND | − | − |
| WNT8B | − | − | − | − | − | − | ND | − | − |
| WNT10A | − | − | − | − | − | − | ND | − | − |
| WNT10B | + | + | + | + | − | − | − | + | − |
| WNT11 | − | − | − | − | − | − | ND | + | − |
| WNT14 | − | − | − | + | − | − | − | + | − |
| FRP1 | − | − | − | + | − | + | + | + | + |
| FRP2 | − | − | − | + | − | − | − | + | + |
| FRP3 | − | − | − | − | − | − | − | + | − |
| FRP4 | − | − | − | + | − | − | − | + | + |
| FRP5 | − | − | − | − | − | − | − | + | − |

Formalin- or paraformaldehyde-fixed and paraffin-embedded tissue sections of human RA or OA synovium were deparaffinized, rehydrated in ethanol series, and treated in a microwave oven (500 W, 4 minutes, 3 times) in 0.01 M sodium citrate buffer (pH6.0). After the treatment, the tissue sections was incubated with normal goat serum, normal donkey serum, normal rabbit serum or 1% bovine serum albumin at room temperature for 30 minutes, and with goat anti-Wnt10b antibody (1 μg/ml, Santa Cruz Biotechnology, Santa Cruz, CA, U.S.A.), goat anti-Frp1 antibody (2 μg/ml, Santa Cruz Biotechnology, Santa Cruz, Calif., U.S.A.), or rabbit anti-von Willbrand Factor (vWF) antibody (200-fold dilution, DAKO, Carpinteria, Calif., U.S.A.). And then, incubated with Alexa Fluor 546 anti-rabbit IgG, Alexa Flour 546 anti-goat IgG (Molecular Probes, Eugene, Oreg., U.S.A.), or FITC-labeled anti-goat IgG (Vector, Burlingame, Calif., U.S.A.) at room temperature for 90 minutes, and tissue localization of each antigen was examined by a fluorescene microscope.

In RA synovium, WNT 10B localized in synovial surface cells and endothelial cells. In OA synovium, the reaction was not observed. On the contrary, FRP1 localized in synovial surface cells and endothelial cells in OA tissues, but not in RA tissues.

Localization of WNT10B and FRP1 in endothelial cells was confirmed by a double immunostaining technique using anti-vWF antibody, which specifically recognizes endothelial cells.

From the immunohistostaining micrograms obtained by said staining technique, WNT10B-positive cells in RA synovium and FRP1-positive cell in OA synovium, which confirmed the specific expression pattern in RA and OA synovial tissues, respectively.

### POSSIBILITY FOR THE INDUSTRIAL APPLICABILITY

By the present invention, the profiling of expression pattern of WNTs and FRPs in RA and OA synovial tissues was clarified. That is, by RT-PCR analysis using gene-specific primer sets, especially, by the positive and/or negative of amplification of the genes by RT-PCR analysis for WNT10B or combinatorial detection of WNT10B and FRP1, the early RA specific diagnosis without false-positive reaction is accomplished. Accordingly, easy and reliable RA-specific diagnosis can be accomplished, and can provide an excellent technique which has high industrial applicability.

1. A method to detect rheumatoid arthritis by detecting at least the upregulation of expression of WNT10B in joint synovial fluid, in joint synovial tissue or in peripheral blood.

2. The method to detect rheumatoid arthritis of claim 1, wherein at least the upregulation of expression of WNT10B is detected by RT-PCR analysis.

3. The method to detect rheumatoid arthritis of claim 1, wherein at least inhabitation of expression of FRP is detected in parallel.

4. The method to detect rheumatoid arthritis of claim 2, wherein at least an inhabitation of expression of FRP is detected in parallel.


SEQUENCE LISTING (44 sequences)

SEQ ID NO: 1 - DNA, 20 bp, Artificial Sequence
  primer
tcctgctcagaaggttccat

SEQ ID NO: 2 - DNA, 20 bp, Artificial Sequence
  primer
gctgtacgtgcagaagttgg

SEQ ID NO: 3 - DNA, 20 bp, Artificial Sequence
  primer
ctgtatcagggaccgagagg

SEQ ID NO: 4 - DNA, 20 bp, Artificial Sequence
  primer
caaagagaactcgccaggag

SEQ ID NO: 5 - DNA, 20 bp, Artificial Sequence
  primer
actgagtgtgtgcagctgtg

SEQ ID NO: 6 - DNA, 20 bp, Artificial Sequence
  primer
tgatgtcttgctgcagacac

SEQ ID NO: 7 - DNA, 20 bp, Artificial Sequence
  primer
acttcggcgtgttagtctcc

SEQ ID NO: 8 - DNA, 20 bp, Artificial Sequence
  primer
atttttccttccgcttctcc

SEQ ID NO: 9 - DNA, 20 bp, Artificial Sequence
  primer
ttgaggagtgccactaccag

SEQ ID NO: 10 - DNA, 20 bp, Artificial Sequence
  primer
ttgaactgtgcgttgcgtgg

SEQ ID NO: 11 - DNA, 20 bp, Artificial Sequence
  primer
cagttcaagaccgtgcagac

SEQ ID NO: 12 - DNA, 20 bp, Artificial Sequence
  primer
tggaacctacccatcccata

SEQ ID NO: 13 - DNA, 20 bp, Artificial Sequence
  primer
gtgctgcttcgtcaggtgta

SEQ ID NO: 14 - DNA, 20 bp, Artificial Sequence
  primer
cgaggttgaagctgagttcc

SEQ ID NO: 15 - DNA, 20 bp, Artificial Sequence
  primer
caactgcacaacaacgaggc

SEQ ID NO: 16 - DNA, 20 bp, Artificial Sequence
  primer
gtactacgcagcaccagtgg

SEQ ID NO: 17 - DNA, 20 bp, Artificial Sequence
  primer
gagaagcaaggccagtacca

SEQ ID NO: 18 - DNA, 20 bp, Artificial Sequence
  primer
acagcacatgaggtcacagc

SEQ ID NO: 19 - DNA, 20 bp, Artificial Sequence
  primer
acatgctatcagctctgctg

SEQ ID NO: 20 - DNA, 20 bp, Artificial Sequence
  primer
aaagatcagttccgcctctg

SEQ ID NO: 21 - DNA, 20 bp, Artificial Sequence
  primer
gaaagtggcaagctttggag

SEQ ID NO: 22 - DNA, 20 bp, Artificial Sequence
  primer
gaaagtggcaagctttggag

SEQ ID NO: 23 - DNA, 20 bp, Artificial Sequence
  primer
aatgaggcttcacaacaacc

SEQ ID NO: 24 - DNA, 20 bp, Artificial Sequence
  primer
tcatgtggtccaatctcctc

SEQ ID NO: 25 - DNA, 20 bp, Artificial Sequence
  primer
cttcattgatacccacaacc

SEQ ID NO: 26 - DNA, 20 bp, Artificial Sequence
  primer
attgttggggagaaggctac

SEQ ID NO: 27 - DNA, 20 bp, Artificial Sequence
  primer
tgacctcaagacccgatacc

SEQ ID NO: 28 - DNA, 20 bp, Artificial Sequence
  primer
caagtgaaggcaaagcacaa

SEQ ID NO: 29 - DNA, 20 bp, Artificial Sequence
  primer
aagatggtgccaacttcacc

SEQ ID NO: 30 - DNA, 20 bp, Artificial Sequence
  primer
taaggaaccagccaggacac

SEQ ID NO: 31 - DNA, 20 bp, Artificial Sequence
  primer
gtgacaccaccttgcagaac

SEQ ID NO: 32 - DNA, 20 bp, Artificial Sequence
  primer
accctctgatgtacggttgc

SEQ ID NO: 33 - DNA, 20 bp, Artificial Sequence
  primer
cttgttcttgcagcattccc

SEQ ID NO: 34 - DNA, 20 bp, Artificial Sequence
  primer
agagaaggcaatgcctctcc

SEQ ID NO: 35 - DNA, 20 bp, Artificial Sequence
  primer
aaagacagcttgcagtgcac

SEQ ID NO: 36 - DNA, 20 bp, Artificial Sequence
  primer
tgttatgacaacctcagtgg

SEQ ID NO: 37 - DNA, 20 bp, Artificial Sequence
  primer
cattgacttccagcacgagc

SEQ ID NO: 38 - DNA, 20 bp, Artificial Sequence
  primer
acgaagcttcatatcccagc

SEQ ID NO: 39 - DNA, 20 bp, Artificial Sequence
  primer
agaggagtggctgcaatgag

SEQ ID NO: 40 - DNA, 20 bp, Artificial Sequence
  primer
tggccttacataggctgtcc

SEQ ID NO: 41 - DNA, 20 bp, Artificial Sequence
  primer
aagtggatggacagctgctg

SEQ ID NO: 42 - DNA, 20 bp, Artificial Sequence
  primer
tactttctgagaccctgagg

SEQ ID NO: 43 - DNA, 20 bp, Artificial Sequence
  primer
gtcagtggtggacctgacct

SEQ ID NO: 44 - DNA, 20 bp, Artificial Sequence
  primer
aggggagcttcagtgtggtg